(19) United States
(12) Patent Application Publication
(10) Pub. No.: US 2022/0257912 A1
(43) Pub. Date: Aug. 18, 2022

(54) LAMINATION APPARATUS FOR MEDICAL BALLOONS AND RELATED METHODS

(71) Applicant: C.R. BARD, INC., Tempe, AZ (US)

(72) Inventors: Melissa Boyle, Phoenix, AZ (US); Clint Solomon, Gilbert, AZ (US); Edgar Sanchez-Garcia, Tempe, AZ (US); Claire Millar, Phoenix, AZ (US)

(21) Appl. No.: 17/625,417

(22) PCT Filed: Jul. 19, 2019
(86) PCT No.: PCT/US2019/042576

§ 371 Date: Jan. 7, 2022

(51) Int. Cl. A61M 25/10

(52) U.S. Cl. CPC A61M 25/1029; A61M 25/1018

(57) ABSTRACT

An apparatus (10) for laminating a medical balloon (12). In one embodiment, the lamination apparatus includes a pressurized medical balloon (12) comprising a plurality of layers in need of lamination. The balloon is positioned in an evacuated, flexible receiver (14) in which the pressurized medical balloon is positioned, and heat may be applied to the balloon to cause consolidation of one or more of the layers and result in lamination A fixture is also provided for simultaneously laminating a plurality of balloons.

### INCORPORATION BY REFERENCE

All publications and patent applications mentioned in this specification are herein incorporated by reference to the same extent as if each individual publication or patent application was specifically and individually indicated to be incorporated by reference.

### TECHNICAL FIELD

This disclosure pertains to the manufacture of devices for providing medical treatment and, in particular, an apparatus and related methods for manufacturing a medical balloon.

### BACKGROUND

Balloon dilatation catheters are used to treat lesions in vessels, such as by way of angioplasty. Forming each balloon typically involves a consolidation process, which may be achieved by blowing a parison (which is typically formed of multiple layers in need of lamination) into a metal die. As can be appreciated, this requires that the die precisely correspond in size and shape to the desired finished product, which necessitates having multiple dies to manufacture a variety of shapes and sizes of balloons, and a corresponding increase in complexity and cost (including in terms of process validation). Furthermore, inaccurate sizing of the parison to the particular die can result in uneven or incomplete contact with the die, resulting in uneven or inconsistent lamination of the resulting balloon, which can lead to delamination, deflation, or reduced burst pressure. A single die also may handle only one balloon at a time, making this step in the balloon catheter manufacturing process rather slow and tedious.

Accordingly, it would be desirable to provide a simple, yet effective apparatus and method for consolidating or laminating a medical balloon. The apparatus and method would operate on multiple sizes or shapes of medical balloons without modification or added operational cost, and would be more efficient that past approaches, including by allowing for the simultaneous processing of multiple medical balloons to achieve consolidation or lamination.

### SUMMARY

An object of the invention is to provide an apparatus laminating an object, such as a medical balloon, that avoids the need for a die having a fixed size and dimension, and uses positive pressure to inflate the medical balloon during the lamination process. In one embodiment, the apparatus comprises a pressurized medical balloon comprising a plurality of layers in need of lamination, and an evacuated, flexible receiver in which the pressurized medical balloon is positioned.

In some embodiments, an inflator is provided for inflating the pressurized medical balloon. The inflator may be connected to the pressurized medical balloon in the evacuated, flexible receiver. One or more seals may be provided for sealing the pressurized medical balloon.

In some embodiments, the evacuated, flexible receiver comprises a bag. The evacuated, flexible receiver may comprise opposed sheets of material between which the pressurized balloon is located. Each sheet of material may be connected to a frame forming part of a fixture. A plurality of pressurized balloons are located between the opposed sheets of material, and each balloon may be connected to a pressurized fluid supply, such as a manifold.

In some embodiments, a heater is provided for heating the pressurized medical balloon. The heater may comprise a convective heater or a radiant heater. The heater may comprise a heated pressure chamber for receiving the pressurized medical balloon in the evacuated, flexible receiver.

According to a further aspect of the disclosure, a lamination apparatus comprises a fixture including a flexible receiver forming a chamber for receiving and inflating a plurality of medical balloons having a plurality of layers in need of lamination, the chamber adapted for being evacuated. In some embodiments, the fixture includes at least one manifold for supplying pressurized fluid to each of the plurality of medical balloons. The fixture may include a first frame portion including a first flexible sheet, and a second frame portion including a second flexible sheet, the first and second flexible sheets forming the flexible receiver.

According to another aspect of the disclosure, a method of laminating a medical balloon having a plurality of layers in need of lamination is provided. The method comprises placing the medical balloon into a flexible receiver. The method further comprises pressurizing the medical balloon, and evacuating the flexible receiver.

In some embodiments, the method includes heating the pressurized medical balloon in the evacuated flexible receiver. The heating step may comprise placing the evacuated flexible receiver into an oven. In some embodiments, the step of pressurizing the medical balloon is completed after the placing step. In some embodiments, the step of pressurizing the medical balloon is completed before the placing step.

Yet a further aspect of the disclosure pertains to a method of laminating a plurality of medical balloons, each medical balloon having a plurality of layers in need of lamination. The method comprises placing the plurality of medical balloons into a single receiver. The method further comprises pressurizing the plurality of medical balloons and evacuating the single receiver.

In some embodiments, the method further includes the step of heating the plurality of pressurized medical balloons in the single evacuated receiver. In some embodiments, the evacuating step is performed after the pressurizing step. In some embodiments, the pressurizing step is performed prior to the placing step.

### BRIEF DESCRIPTION OF THE DRAWING FIGURES

The above and further advantages of the invention according to the disclosure may be better understood by referring to the following description in conjunction with the accompanying drawings in which:

FIG. 1 schematically illustrates a first embodiment of a lamination apparatus including a flexible bag including a pressurized medical balloon having layers in need of lamination;

FIG. 2 illustrates a heater in the form of an oven for receiving the flexible bag including the pressurized medical balloon;

FIG. 3 schematically illustrates a second embodiment of a lamination apparatus including a flexible bag including a pressurized medical balloon having layers in need of lamination, with the pressurization provided by a constant supply of pressurized fluid;

FIG. 4 is a perspective view of another lamination apparatus specially designed for simultaneously laminating a plurality of objects, such as medical balloons;

FIG. 5 is a top view of the apparatus of FIG. 4; and

FIGS. 6 and 7 illustrate the operation of the apparatus of FIGS. 4 and 5.

The drawings are not necessarily drawn proportionally or to scale. For example, the dimensions of some of the elements may be exaggerated relative to other elements for clarity or several physical components may be included in one functional block or element. Further, sometimes reference numerals may be repeated among the drawings to indicate corresponding or analogous elements.

### DETAILED DESCRIPTION

In the following detailed description, numerous specific details are set forth to provide a thorough understanding of the disclosed concepts. Those of ordinary skill in the art will know that the disclosed inventions may be practiced without these specific details. In other instances, well-known methods, procedures, components, or structures may not have been described in detail so as not to obscure the disclosed inventions.

The description provided below applies to all embodiments unless noted otherwise, and features common to each embodiment are similarly shown and numbered.

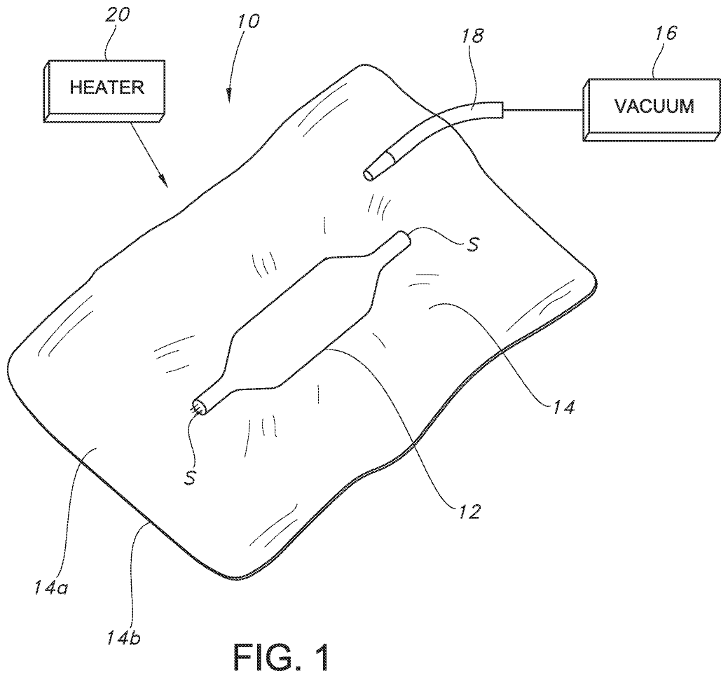

Referring first to FIG. 1, one embodiment of a lamination apparatus 10 for laminating objects in need thereof is illustrated. In this embodiment, the apparatus 10 comprises a pressurized medical balloon 12 including a plurality of layers in need of lamination. The balloon 12 for medical use is typically of the form illustrated, with two tapering ends, or “cones,” terminating in necks with openings, and an intermediate cylindrical or “barrel” section. However, the particular application of the disclosed apparatus 10 is not considered to be limited to any particular type of balloon.

Regardless of the size and shape, the balloon 12 may include any number of layers, which can comprise any suitable material. Typical layers may include, for example, an inner layer comprising a polymer film, one or more fiber layers, and an outer polymer film layer, which layers may include an adhesive or bonding agent. By “pressurized,” it is meant that the balloon is inflated with an inflation fluid, which may be achieved by applying the inflation fluid to the balloon to inflate it, and then sealing the end(s), or in a continuous manner, as outlined further in the following description.

The apparatus 10 further comprises a flexible receiver in which the pressurized medical balloon 12 is positioned. In the illustrated embodiment, the flexible receiver comprises a bag 14 for receiving the pressurized balloon 12. The bag 14 may comprise two flexible sheets 14a, 14b of polymer material arranged in an opposed relationship sealed together (such as by a thermal weld formed by a welder, such as a bar sealer or the like). The bag 14 thus includes an interior compartment in which the balloon 12 is positioned prior to lamination. For purposes of illustration, these sheets may be considered upper and lower panels of the bag 14, although the particular orientation is not considered relevant.

In the illustrated embodiment, the balloon 12 is pressurized and the two open ends of it are sealed, such as by using a seal S (which may take the form of a plug), prior to placement in the bag 14. Once the balloon 12 is located in the bag, the bag 14 is sealed and then evacuated, such as by connecting the interior compartment with an external source of negative pressure, such as vacuum 16, using a conduit or tube 18. This causes the upper and lower sheets 14a, 14b of the bag 14 to compress against the outer surfaces of the balloon 12, which as a result of the internal pressurization causes any layers in the wall thereof to be compressed.

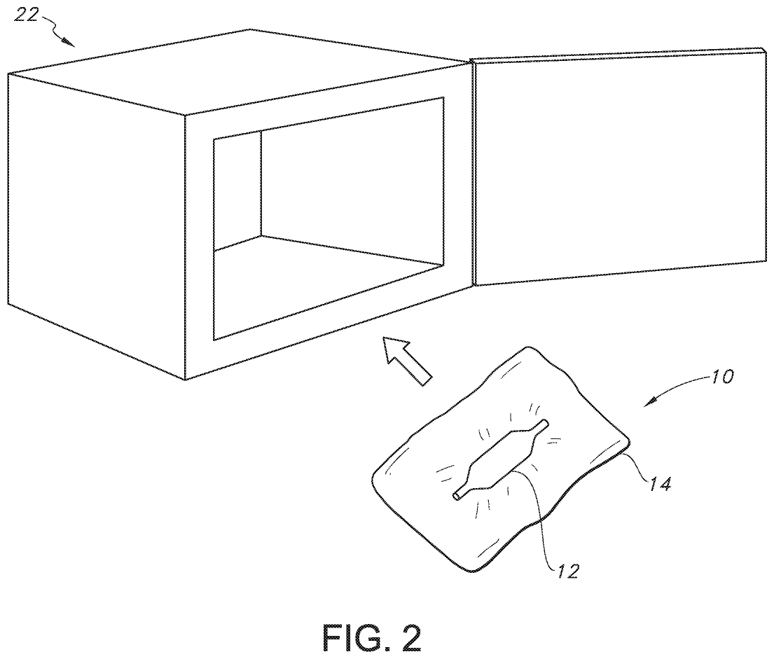

With the compressive force applied, the balloon 12 is caused to become laminated, such as for example by the distribution of an adhesive between the layers. Optionally, but in most cases desirably, the balloon 12 may further be subjected to heating (with or without the connection to conduit 18, which may be removed and the bag 14 fully sealed in an evacuated state). This heating may be achieved by applying heat directly to the bag 14, such as by using a heater 20 (such as a heat lamp (radiation), hot air (convection), hot water bath (convection), or any combination of the foregoing applied simultaneously or in series)) also forming part of the apparatus 10. Alternatively, the evacuated bag 14 including the pressurized balloon 12 may be placed into a heater in the form of an oven 22, as shown in FIG. 2. Regardless of the approach, the particular temperature used may vary depending on the nature of the materials used, but would normally be at a temperature and time sufficient to cause a degree of melting and flowing of the materials in order to cause lamination in an even manner.

Once the lamination process is completed, the bag 14 may be opened to retrieve the now-laminated balloon 12. Given the use of fluid pressure to provide the internal resistance to compression by the flexible receiver or bag 14, the balloon 12 may be unsealed to relieve any internal pressure quickly, and without the need for removing any mandrel or the like. A catheter shaft (not shown) may then be applied to the balloon 12 to form a completed balloon catheter, ready for use.

As can be appreciated, the dimensions of the bag 14 may be greater than the dimensions of the largest balloon contemplated, without substantially impacting the ability to provide adequate forces to consolidate the layers. Thus, the bag 14 may be used with a variety of balloon sizes and shapes, thereby avoiding the issues mentioned above stemming from the use of particular metal dies for forming each size and shape of balloon. Moreover, as will be described in more detail below, the proposed technology allows for the simultaneous lamination of a plurality of balloons (including possibly of different sizes and shapes) in a single process to achieve a degree of efficiency that cannot possibly be realized using individual metal dies.

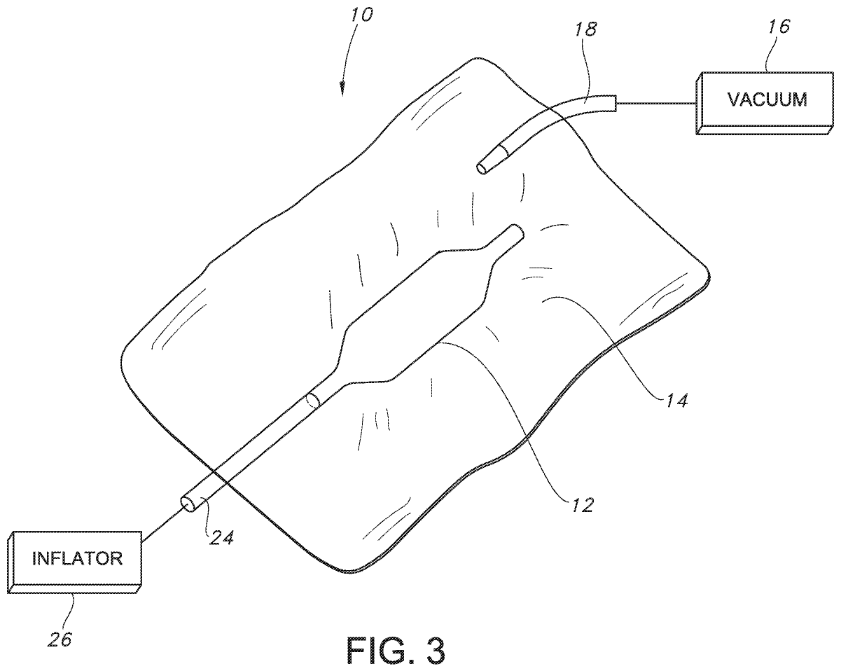

Alternate approaches to the above are envisioned. For instance, instead of pressurizing and sealing the balloon 12 prior to insertion into the bag 14, the balloon may be connected at one end via a conduit 24 to a source of positive fluid pressure, such as an inflator 26, while positioned in the interior compartment of the bag. This is shown in FIG. 3. The other open end of the balloon 12, if present, may be pre-sealed to allow for sufficient positive pressure to be developed and continuously maintained as a result of the connection to the inflator 26. The bag 14 may be evacuated using another conduit 18 communicating with a vacuum 16, and heat applied (by placing the assembly into an oven (not shown) to cause the consolidation and thus lamination to occur. When completed, the bag 14 is opened and the pressure relieved on balloon 12 for immediate use.

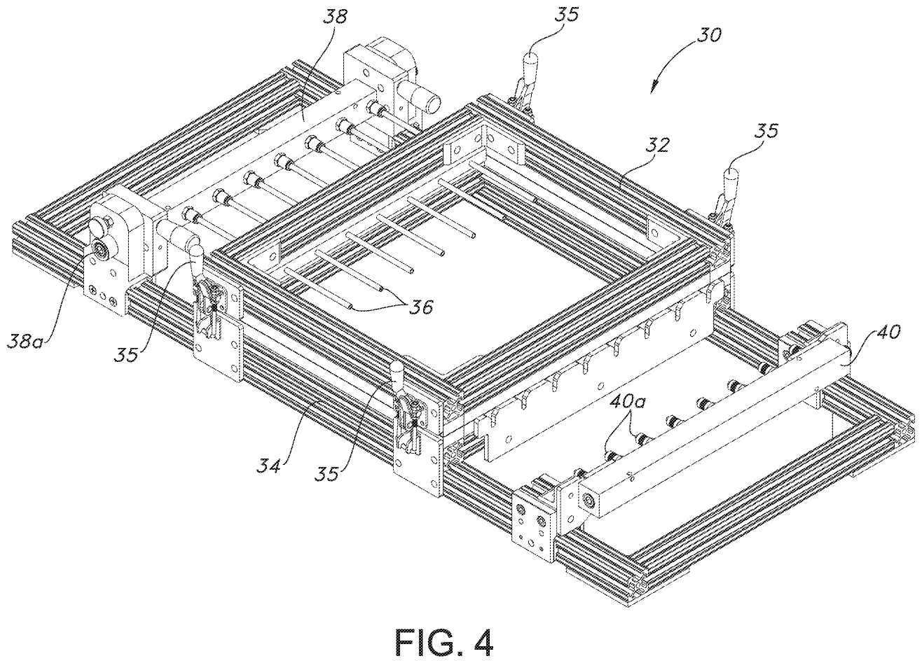

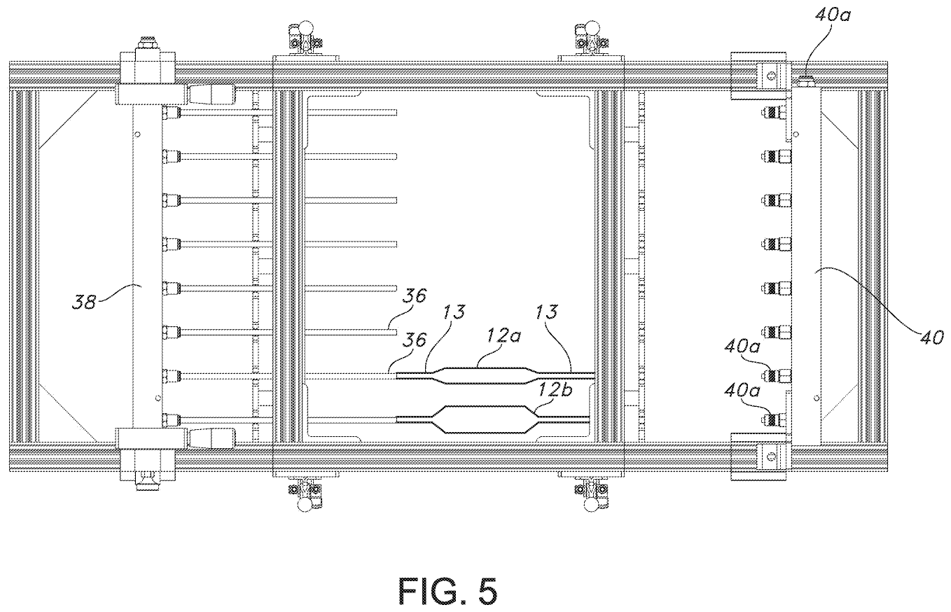

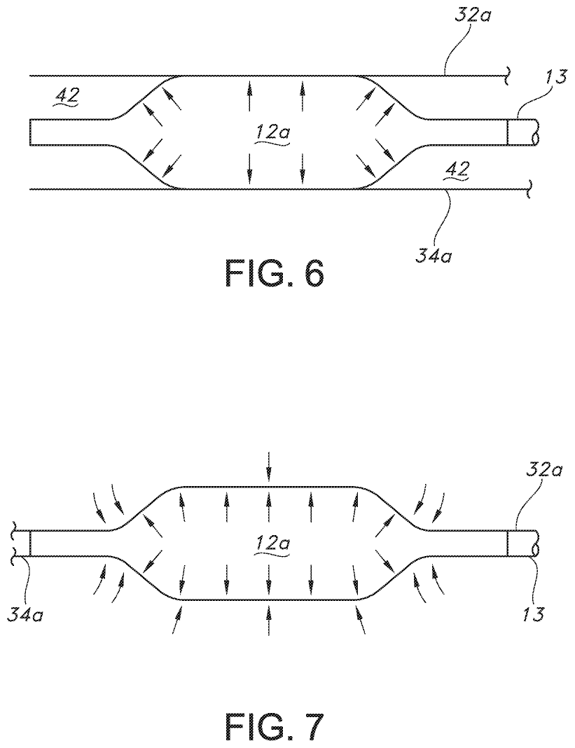

As noted above, the apparatus 10 may be adapted for simultaneously processing (laminating) multiple balloons, even if of varied shapes or sizes. While this can be achieved using the above-described bag technology, FIGS. 4 and 5 illustrate an apparatus in the form of a balloon laminating fixture 30 adapted for receiving and simultaneously laminating a plurality of balloons 12a, 12b. The fixture 30 includes a removable first or upper frame portion 32, which may include a flexible panel or sheet 32a, and a mating second lower frame portion 34, also with a flexible panel or sheet 34a (see FIGS. 6 and 7). Together, the sheets 32a, 34a thus form a flexible receiver, similar in function to bag 14, even though it may include portions such as members of frame portions 32, 34 that make the overall assembly partially rigid. Releasable clamps 35 may also be provided for providing a compressive force for sealing the frame portions 32, 34.

The frame portions 32, 34 when mated may be sealed together over a plurality of balloons 12a, 12b located therein (which may be different sizes and shapes), as shown in FIG. 5. The balloons 12a, 12b may be supported by individual conduits 36 in communication with a vacuum manifold 38 at one end, via tubular shafts 13 (which are supported by, but not in fluid communication with the conduits), and allow for negative pressure to be maintained within the chamber thus formed. These shafts 13 may extend beyond the sealed frame portions 32, 34 at one end, and suitable gasketing or seals may be provided to ensure an airtight connection is created. The opposite ends of the shafts 13 may be coupled to ports 40a on an inflation manifold 40 the corresponding end. The manifold 40 may be used to supply inflation fluid to the balloons 12 simultaneously, such as by valves 38, 40a connected to a pressurized fluid supply (not shown)

The portions 32, 34 when sealed together thus form a compartment of chamber 42 between panels or sheets 32a, 34a, as indicated schematically in FIG. 6. This chamber 42 may be evacuated by applying negative pressure via manifold 38 and conduits 36 to cause the panels or sheets 32a, 34a to compress together over the balloons 12a 12b, as indicated in FIG. 7. The positive pressure supplied to balloons 12a, 12b via manifold 40 concurrently provides outward radial resistance to the externally applied compressive force.

Heat may then be applied to complete the lamination process, such as by placing the entire fixture 30 into an oven. After a suitable time at a given temperature, which again may vary depending on the material properties, the heat is removed, and the portions 32, 34 opened to simultaneously recover a plurality of laminated balloons formed during a single processing, and avoiding the need for specialized metal dies for each size and shape of balloon. As can be appreciated, a plurality of fixtures, each including a plurality of balloons, may be simultaneously pressurized and heated in a single oven to further enhance productivity.

Still another option is to place the pressurized balloon(s) within evacuated bag 14 or chamber 42 formed by sheets 32a, 34a of fixture 30 into a pressurized, heated chamber. This chamber, which may be similar to oven but adapted to be positively pressurized as well as for applying heat (convection, radiation, or both), may supply the external pressure and heat simultaneously for assisting in consolidating the balloon layers.

This disclosure may be considered to relate to the following items:

1. A lamination apparatus, comprising:

a pressurized medical balloon comprising a plurality of layers in need of lamination; and

an evacuated, flexible receiver in which the pressurized medical balloon is positioned.

2. The lamination apparatus of item 1, further including an inflator for inflating the pressurized medical balloon.
3. The lamination apparatus of item 2, wherein the inflator is connected to the pressurized medical balloon in the evacuated, flexible receiver.
4. The lamination apparatus of any of the foregoing items, further including a seal for sealing the pressurized medical balloon.
5. The lamination apparatus of any of the foregoing items, wherein the evacuated, flexible receiver comprises a bag.
6. The lamination apparatus of any of the foregoing items, wherein the evacuated, flexible receiver comprises opposed sheets of material between which the pressurized balloon is located.
7. The lamination apparatus of item 6, wherein each sheet of material connected to a frame.
8. The lamination apparatus of item 6, wherein a plurality of pressurized balloons are located between the opposed sheets of material
9. The lamination apparatus of item 7, wherein each balloon is connected to a pressurized fluid supply.
10. The lamination apparatus of item 9, wherein the pressurized fluid supply comprises a manifold.
11. The lamination apparatus of any of the foregoing items, further including a heater for heating the pressurized medical balloon.
12. The lamination apparatus of item 11, wherein the heater comprises a convective heater.
13. The lamination apparatus of item 12, wherein the heater comprises a radiant heater.
14. The lamination apparatus of any of the foregoing items, further including a heated pressure chamber for receiving the pressurized medical balloon in the evacuated, flexible receiver.
15. A lamination apparatus, comprising:

a fixture including a flexible receiver forming a chamber for receiving and inflating a plurality of medical balloons having a plurality of layers in need of lamination, the chamber adapted for being evacuated.

16. The lamination apparatus of item 15, wherein the fixture includes at least one manifold for supplying pressurized fluid to each of the plurality of medical balloons.
17. The lamination apparatus of item 15 or item 16, wherein the fixture includes a first frame portion including a first flexible sheet, and a second frame portion including a second flexible sheet, the first and second flexible sheets forming the flexible receiver.
18. A method of laminating a medical balloon having a plurality of layers in need of lamination, comprising:

placing the medical balloon into a flexible receiver;

pressurizing the medical balloon; and

evacuating the flexible receiver.

19. The method of item 18, further including the step of heating the pressurized medical balloon in the evacuated flexible receiver.
20. The method of item 18, wherein the heating step comprises placing the evacuated flexible receiver into an oven.
21. The method of any of items 18-20, wherein the step of pressurizing the medical balloon is completed after the placing step.
22. The method of any of items 18-20, wherein the step of pressurizing the medical balloon is completed before the placing step.
23. A method of laminating a plurality of medical balloons, each medical balloon having a plurality of layers, comprising:

placing the plurality of medical balloons into a single receiver;

pressurizing the plurality of medical balloons; and

evacuating the single receiver.

24. The method of item 23, further including the step of heating the plurality of pressurized medical balloons in the single evacuated receiver.
25. The method of item 23 or item 24, wherein the evacuating step is performed after the pressurizing step.
26. The method of item 23 or item 24, wherein the pressurizing step is performed prior to the placing step.

Each of the following terms written in singular grammatical form: “a”, “an”, and the”, as used herein, means “at least one”, or “one or more”. Use of the phrase One or more” herein does not alter this intended meaning of “a”, “an”, or “the”. Accordingly, the terms “a”, “an”, and “the”, as used herein, may also refer to, and encompass, a plurality of the stated entity or object, unless otherwise specifically defined or stated herein, or the context clearly dictates otherwise. For example, the phrases: “a unit”, “a device”, “an assembly”, “a mechanism”, “a component, “an element”, and “a step or procedure”, as used herein, may also refer to, and encompass, a plurality of units, a plurality of devices, a plurality of assemblies, a plurality of mechanisms, a plurality of components, a plurality of elements, and, a plurality of steps or procedures, respectively.

Each of the following terms: “includes”, “including”, “has”, “having”, “comprises”, and “comprising”, and, their linguistic/grammatical variants, derivatives, or/and conjugates, as used herein, means “including, but not limited to”, and is to be taken as specifying the stated components), feature(s), characteristic(s), parameter(s), integer(s), or step(s), and does not preclude addition of one or more additional component(s), feature(s), characteristic(s), parameter(s), integer(s), step(s), or groups thereof. Each of these terms is considered equivalent in meaning to the phrase “consisting essentially of.” Each of the phrases “consisting of” and “consists of, as used herein, means “including and limited to”. The phrase “consisting essentially of means that the stated entity or item (system, system unit, system sub-unit device, assembly, sub-assembly, mechanism, structure, component element or, peripheral equipment utility, accessory, or material, method or process, step or procedure, sub-step or sub-procedure), which is an entirety or part of an exemplary embodiment of the disclosed invention, or/and which is used for implementing an exemplary embodiment of the disclosed invention, may include at least one additional feature or characteristic” being a system unit system subunit device, assembly, sub-assembly, mechanism, structure, component or element or, peripheral equipment utility, accessory, or material, step or procedure, sub-step or sub-procedure), but only if each such additional feature or characteristic” does not materially alter the basic novel and inventive characteristics or special technical features, of the claimed item.

The term “method”, as used herein, refers to steps, procedures, manners, means, or/and techniques, for accomplishing a given task including, but not limited to, those steps, procedures, manners, means, or/and techniques, either known to, or readily developed from known steps, procedures, manners, means, or/and techniques, by practitioners in the relevant field(s) of the disclosed invention.

Terms of approximation, such as the terms about, substantially, approximately, etc., as used herein, refer to ±10% of the stated numerical value.

It is to be fully understood that certain aspects, characteristics, and features, of the invention, which are, for clarity, illustratively described and presented in the context or format of a plurality of separate embodiments, may also be illustratively described and presented in any suitable combination or sub-combination in the context or format of a single embodiment. Conversely, various aspects, characteristics, and features, of the invention which are illustratively described and presented in combination or sub-combination in the context or format of a single embodiment may also be illustratively described and presented in the context or format of a plurality of separate embodiments.

Although the invention has been illustratively described and presented by way of specific exemplary embodiments, and examples thereof, it is evident that many alternatives, modifications, or/and variations, thereof, will be apparent to those skilled in the art. Accordingly, it is intended that all such alternatives, modifications, or/and variations, fall within the spirit of, and are encompassed by, the broad scope of the appended claims.

1. A lamination apparatus, comprising:
a pressurized medical balloon comprising a plurality of layers in need of lamination; and
an evacuated, flexible receiver in which the pressurized medical balloon is positioned.

2. The lamination apparatus of claim 1, further including an inflator for inflating the pressurized medical balloon.

3. The lamination apparatus of claim 2, wherein the inflator is connected to the pressurized medical balloon in the evacuated, flexible receiver.

4. The lamination apparatus of claim 1, further including a seal for sealing the pressurized medical balloon.

5. The lamination apparatus of claim 1, wherein the evacuated, flexible receiver comprises a bag.

6. The lamination apparatus of claim 1, wherein the evacuated, flexible receiver comprises opposed sheets of material between which the pressurized balloon is located.

7. The lamination apparatus of claim 6, wherein each sheet of material connected to a frame.

8. The lamination apparatus of claim 6, wherein a plurality of pressurized balloons are located between the opposed sheets of material

9. The lamination apparatus of claim 7, wherein each balloon is connected to a pressurized fluid supply.

10. The lamination apparatus of claim 9, wherein the pressurized fluid supply comprises a manifold.

11. The lamination apparatus of claim 1, further including a heater for heating the pressurized medical balloon.

12. The lamination apparatus of claim 11, wherein the heater comprises a convective heater.

13. The lamination apparatus of claim 12, wherein the heater comprises a radiant heater.

14. The lamination apparatus of claim 1, further including a heated pressure chamber for receiving the pressurized medical balloon in the evacuated, flexible receiver.

15. A lamination apparatus, comprising:
a fixture including a flexible receiver forming a chamber for receiving and inflating a plurality of medical balloons having a plurality of layers in need of lamination, the chamber adapted for being evacuated.

16. The lamination apparatus of claim 15, wherein the fixture includes at least one manifold for supplying pressurized fluid to each of the plurality of medical balloons.

17. The lamination apparatus of claim 15, wherein the fixture includes a first frame portion including a first flexible sheet, and a second frame portion including a second flexible sheet, the first and second flexible sheets forming the flexible receiver.

18. A method of laminating a medical balloon having a plurality of layers in need of lamination, comprising:
placing the medical balloon into a flexible receiver;
pressurizing the medical balloon; and
evacuating the flexible receiver.

19. The method of claim 18, further including the step of heating the pressurized medical balloon in the evacuated flexible receiver.

20. The method of claim 18, wherein the heating step comprises placing the evacuated flexible receiver into an oven.

21.-26. (canceled)
